(19) United States
(12) Patent Application Publication
(10) Pub. No.: US 2020/0038437 A1
(43) Pub. Date: Feb. 6, 2020

(54) Base Medicinal Formulation for Supporting the Specific Homeostatic Acid-Base Balance of Differentiated Living Tissues

(71) Applicant: Lawrence M. Green, Miami, FL (US)

(72) Inventor: Lawrence M. Green, Miami, FL (US)

(21) Appl. No.: 16/055,076

(22) Filed: Aug. 4, 2018

(51) Int. Cl. A61K 33/10; A61K 9/00; A61K 33/00; A61K 33/30

(52) U.S. Cl. CPC A61K 33/10; A61K 9/0053; A61K 33/30; A61K 33/00; A61K 9/0019

(57) ABSTRACT

This invention is a base medicinal formulation that supports autonomic metabolic mechanisms for maintaining the specific homeostatic acid-base balance of differentiated living tissues in a body by influencing alterations in the anion gap and transitionally buffering acid in support of pH balance in arterial blood plasma in the narrow alkaline range of 7.37 to 7.43.

### BACKGROUND OF THE INVENTION

This invention relates to a base medicinal formulation that delivers essential and indispensable base minerals that influence alterations in the anion gap as sacrificial bio-anodes and bio-cathodes in intracellular fluids, extracellular fluids and all other differentiated living tissues in the human body. This base medicinal formulation also delivers a transitional base chemical of the metabolic process that buffers acid in support of pH balance in arterial blood plasma in the narrow alkaline range of 7.37 to 7.43. The formulation of this invention consists of potassium bicarbonate [KHCO₃⁻], sodium bicarbonate [NaHCO₃⁻], magnesium carbonate [MgCO₃] and zinc carbonate [ZnCO₃] within specific ranges expressed in milligrams and percentages. These inorganic compounds, selected for their unique combined chemical and electrochemical properties, are present together in the invention to support autonomic metabolic mechanisms that allow differentiated living tissues in a body to adjust serum anion gap levels and maintain their specific homeostatic acid-base equilibrium when bicarbonate production is insufficient or endogenous acid production overwhelms the pathophysiological rate and the body is using alkaline minerals sacrificed from physiological repositories.

The formulation of this invention can be consumed orally in dosage units formed as tablets, capsules, gelcaps, caplets and other ingestible delivery mechanisms. These ingestible delivery mechanisms are enteric-coated to permit their transport through the stomach intact, becoming dissolvable in the small intestines. These delivery mechanisms also include a controlled-release agent wherein the formulation's inorganic compounds are control-released from their ingestible delivery mechanisms into the small intestines and are absorbed by the arterial blood supply as bioavailable active ingredients that assimilate into intracellular fluids, extracellular fluids and all other differentiated living tissues in a body.

The invention also can be dissolved in a solution of United States Pharmacopeia (“USP”) purified pharmaceutical grade water and administered intravenously in standard dosage units. The intravenous routes of administration include both infusion (commonly referred to as “drips”) and injection (commonly referred to as “sharps”). Infusion introduces the formulation (dissolved in a solution of USP purified pharmaceutical grade water) into a vein. Injection introduces the formulation (dissolved in USP purified pharmaceutical grade water) into a vein, an artery or other targeted differentiated living tissues in the human body.

Arterial blood plasma within the narrow alkaline range of 7.37 to 7.43 pH is a basic requirement of human health. Maintenance of homeostatic acid-base balance in the arterial blood supply, intracellular fluids, extracellular fluids and all other differentiated living tissues in a body is indispensable to metabolic function. Minute fluctuations in arterial blood pH are clinically significant. When bicarbonate production is insufficient or endogenous acid production (“EAP”) overwhelms the pathophysiological rate, the body resorbs essential alkaline minerals sacrificed from physiologically essential repositories in order to compensate for pH swings (imbalances) caused by EAP or other factors. These swings can occur in days, hours or milliseconds and relatively small alterations in plasma potassium (K), sodium chloride (NaCl), or magnesium (Mg) concentrations (this is the anion gap) can have major clinical manifestations.

Metabolic function in global populations has increasingly come under pressure from widespread adoption of “Western” lifestyles and incidental environmental exposures thereto. The causes of metabolic dysfunction (e.g., metabolic acidosis) include increased consumption, or the endogenous generation, of organic acids, as well as either insufficient production of bicarbonate, or renal and/or gastro-intestinal loss of bicarbonate, such as is seen in renal disease, diarrhœa, pancreatic secretion insufficiencies and biliary fistula. These medical conditions are typically associated with overt laboratory abnormalities: either a frank (acute) acidæmia, a decrease in plasma bicarbonate, and/or an increase in the anion gap.

In comparison, diet-induced ‘low-grade’ metabolic acidosis produces only very small decreases in arterial blood pH and plasma bicarbonate within the range considered to be normal. Within that range, the system equilibrates nearer the lower end of normal rather than the higher end of normal. When the duration of the acidosis is prolonged or chronically present, however, even a low degree of acidosis becomes significant, demonstrably elevating a human body's susceptibility to metabolic dysregulation. This less severe but more chronic “low-grade” acidosis is thought to be brought about primarily by two factors: advancing age with a consequent decline in renal function, and diet (at any age), which may promote acidosis by an increased net acid load, as well as sodium chloride content. The contribution of diet to acidotic influence in the form of net acid load is now well-documented. See e.g., Pizzorno, Joseph, Frassetto, Lynda A., Katzinger, Joseph, “Diet-induced acidosis: is it real and clinically relevant?” British Journal of Nutrition, 103:1187-1188 (2010). With age, the severity of diet-dependent acidosis increases independently of the diet, most likely due to a decline in kidney function and capacity. Renal insufficiency, a disease state complication manifested at any age, contributes to metabolic acidosis by reducing conserved filtered bicarbonate and the excretion of acid.

Prolonged or chronically present disturbances of homeostatic acid-base equilibrium in differentiated living tissues are also clinically significant because they cause a body to deplete its repositories of vital resources—including essential and indispensable alkaline minerals—in order to maintain the specific homeostatic acid-base equilibrium of those living tissues. Autonomic metabolic mechanisms may precipitate deficiencies of mineral resources the body uses in order to maintain other vital metabolic and essential physiological processes. Such mineral deficiencies are known to contribute, as and when they occur, to the loss of mineral bone density and many other potentially chronic or acute pathophysiological dysfunctions. Moreover, dysfunction occurring in specific organs and organ systems caused by the mechanisms of action found in pharmaceutical drugs and other forms of chemical exposure can challenge acid-base equilibrium with the onset of metabolic disorders characterized as either acidæmias (chronic low-grade or acute forms of acidosis) or alkalemias (chronic low-grade or acute forms of alkalosis). See Pham, A. Q., Xu, L. H., Moe, O. W., Drug-Induced Metabolic Acidosis, doi: 10.12688/f1000research.7006.1.eCollection; PMID: 26918138 (2015).

Each year, millions of individuals around the world are diagnosed with early clinical indications of metabolic syndrome—dyslipidemia (abnormal lipids), abdominal obesity, hypertension, glucose intolerance and insulin resistance. These are conditions that generally progress into chronic metabolic, cardio-metabolic, inflammatory and immunological diseases that require lifelong lifestyle changes and pharmacological vigilance to treat. See THE MERCK MANUAL OF DIAGNOSIS AND THERAPY, Acid-Base Regulation and Disorders, pp. 1263-74, 1266 tbl. 157-2 (18th ed. 2006) (correlating acidæmias and alkalemias to incidences of cardiovascular, neurological, respiratory and metabolic disorders). As a consequence, the World Health Organization is now calling diabetes mellitus a world-wide pandemic and cardiovascular disease remains the leading cause of death in Western societies.

### FIELD OF THE INVENTION

The field of the present invention relates broadly to supporting the optimal health of human beings. Minute fluctuations in arterial blood pH are clinically significant and can be mitigated by influencing alterations in the anion gap and by buffering acid in support of arterial blood plasma pH balance in the narrow alkaline range of 7.37 to 7.43.

The formulation of this invention consists of potassium bicarbonate [KHCO₃⁻], sodium bicarbonate [NaHCO₃⁻], magnesium carbonate [MgCO₃] and zinc carbonate [ZnCO₃] within specific ranges expressed in milligrams and percentages. These inorganic compounds are selected for their unique combined chemical and electrochemical properties and are present together in the invention to support autonomic metabolic mechanisms that allow differentiated living tissues in the human body to adjust serum anion gap levels and maintain their specific homeostatic acid-base equilibrium.

The formulation of this invention can be administered orally, intravenously or by injection. The means of oral administration include dosage units formed as tablets, capsules, gelcaps, caplets and other ingestible delivery mechanisms. These ingestible delivery mechanisms are enteric-coated, which allows them to bypass the stomach intact and become dissolvable in the small intestines. The formulation's inorganic compounds are control-released from their ingestible delivery mechanisms into the small intestines and are absorbed by the arterial blood supply as bioavailable active ingredients that assimilate into intracellular fluids, extracellular fluids and all other differentiated living tissues in a body.

The formulation of this invention also can be dissolved in a solution of United States Pharmacopeia (“USP”) purified pharmaceutical grade water and administered intravenously in standard dosage units. The intravenous routes of administration include both infusion and injection. Intravenous administration by infusion (drips) introduces the formulation (dissolved in USP purified pharmaceutical grade water) into a vein, whereas intravenous administration (dissolved in USP purified pharmaceutical grade water) by injection (sharps) introduces the formulation into a vein, an artery or other targeted differentiated living tissues in the human body.

The invention supports healthy human metabolic physiology by responding to alterations in the anion gap and by reducing endogenous acid in arterial blood, which helps to naturally compensate for disturbances in arterial blood pH (buffering) within the healthy range. Normal metabolism produces hydrogen in the form of volatile and fixed acids. External environments−the air and water that we depend on−also have a tendency to acidify our bodies. Our living tissues, are, with few exceptions, base rather than acidic. Differentiations that govern the respective functions of living tissues in a body also determine homeostatic acid-base equilibrium within a range for each differentiated type of living tissue. Acid-base balance in the arterial blood supply (oxygenated blood from the lungs that the heart pumps to every cell in our bodies) is tightly controlled and must remain within the narrow range of 7.37 to 7.43 pH in order to protect delicate structures within the heart's musculature. Healthy human skin, on the other hand, maintains a lower pH value, ranging between 5.0 and 6.0, approximately. The mild acidity of healthy skin, appearing as an acid mantle that forms on the surface layer of skin tissues, enhances the skin's ability to function as a barrier. In contrast, the gastric juices produced in the stomach are highly acidic, having a pH of approximately 1.5 to 3.5.

Potassium bicarbonate [KHCO₃⁻] and sodium bicarbonate [NaHCO ₃⁻] are present in the formulation within specific ranges expressed in milligrams and percentages because they increase plasma concentrations of bicarbonate in arterial blood. Potassium [K] and sodium [Na] dissociate from bicarbonate [HCO₃⁻] and are dispersed through the arterial blood supply and utilized by the body based on physiological demand. Bicarbonate, a transitional base chemical of the metabolic process, buffers excessive endogenous acid and maintains healthy pH balance within the narrow range in the arterial blood supply by dissociating into carbonic acid (H₂CO₃), which further dissociates into water (H₂O) and carbon dioxide (CO₂), which is exhaled.

Magnesium carbonate [MgCO₃] and zinc carbonate [ZnCO₃] are present in the formulation in trace amounts. Carbonate [CO₃-₂] dissociates from the magnesium [Mg] and zinc [Zn] and either remains a salt in the arterial blood supply based on physiological demand, or is dissolved in carbonic acid, which dissociates into water (H₂O) and carbon dioxide (CO₂), which is exhaled. Magnesium [Mg] and zinc [Zn] are essential and indispensable base minerals dispersed through the arterial blood supply as sacrificial bio-anodes and bio-cathodes. These electro-metallic minerals influence alterations in the anion gap, either by donating electrons that will attract positive H⁺ ions from differentiated living tissues on demand (the anodic sacrifice) or by donating protons that will attract negative H⁻ ions from differentiated living tissues on demand (the cathodic sacrifice). Magnesium is dispersed through the arterial blood supply to low sodium environments in a body where it is efficiently reactive; zinc is dispersed to high sodium environments in a body where it is efficiently reactive.

The unique combined chemical and electrochemical properties of potassium bicarbonate [KHCO₃⁻], sodium bicarbonate [NaHCO₃⁻], magnesium carbonate [MgCO₃] and zinc carbonate [ZnCO₃], formulated together in this invention, support autonomic metabolic mechanisms that allow intracellular fluids, extracellular fluids and all other differentiated living tissues in a body to maintain their specific homeostatic acid-base equilibrium, becoming either more base or less base based on physiological demand. These autonomic metabolic mechanisms are designed to correct even subtle disturbances in acid-base balance that can precipitate a chronic dysregulation or an acute event.

The formulation of this invention provides significant clinical efficacy in mitigating physiological effects when bicarbonate production is insufficient, endogenous acid production (EAP) is excessive and the body's autonomic metabolic mechanisms are overwhelmed. Bicarbonate deficiencies cause the body to sacrifice minerals from physiologically essential repositories in order to compensate for pH swings (chronic low-grade metabolic dysregulation) caused by EAP or other factors. The formulation of this invention mitigates these sacrifices so that, for example, the calcium (Ca⁺⁺) repositoried in bone tissue is conserved.

Magnesium and zinc are also transitional elements of the metabolic process whose anodic and cathodic properties are indispensable to the biochemical and electrochemical function that is present in all metabolic and cellular activities (i.e., intracellular communication). Because the electric potential that exists between the inside of a cell and the outside of a cell is the basis for nearly all transactions occurring within a cell, metabolic dysregulation in differentiated living tissues cannot be corrected unless both the electrochemical and biochemical environments are rebalanced and for that, both magnesium and zinc must be present. Anion Gap, Wilczynski, C., M. D., Staros, E. B., M. D. (Mar. 13, 2014); http://emedicine.medscape.com/article/2087291-overview#a4.

The formulation of this invention provides systemic, clinically-measurable superior and unexpected results in influencing acid-base balance over formulations that exclude magnesium and zinc. Whereas bicarbonate is essential to mitigating low buffer (base) concentrations, the electrochemical properties of magnesium and zinc are uniquely efficacious in achieving acid-base equilibrium in the presence of both low buffer and high buffer concentration environments. See §§ 3.2.2 (Buffer concentration), 3.2.3 (pH), A. ter Heijne, et al., Analysis of bio-anode performance through electrochemical impedance spectroscopy, Bioelectrochemistry, http://dx.doi.org/10.1016/j.bioelechem. 2015.04.002 (2015).

The anodic and cathodic activities of magnesium and zinc in the invention are fundamentally defined in terms of function rather than structure. In low buffer concentration environments, magnesium and zinc influence alterations in the anion gap in differentiated living tissues by donating electrons that will attract positive H⁺ ions from the living tissues, sacrificing themselves in the process of making differentiated living tissues more base (alkaline) based on physiological demand. In high buffer concentration environments, magnesium and zinc influence alterations in the anion gap in differentiated living tissues by donating protons that will attract negative H⁻ ions from the living tissues, sacrificing themselves in the process of making differentiated living tissues less base based on physiological demand.

The photographic images of magnesium and zinc anodes, below, analogously depict the sacrificial nature of magnesium and zinc's chemical and electrochemical properties in situ. The zinc anodes had been immersed in salt water.

### SUMMARY OF THE INVENTION

This invention is a base medicinal formulation that delivers essential and indispensable base minerals that influence alterations in the anion gap as sacrificial bio-anodes and bio-cathodes in intracellular fluids, extracellular fluids and all other living tissues in the human body. This base medicinal formulation also delivers a transitional base chemical of the metabolic process that buffers acid in support of pH balance in arterial blood plasma in the narrow alkaline range of 7.37 to 7.43. The formulation consists of potassium bicarbonate [KHCO₃⁻], sodium bicarbonate [NaHCO₃₋], magnesium carbonate [MgCO₃] and zinc carbonate [ZnCO₃] within specific ranges expressed in milligrams and percentages. These inorganic compounds are selected for their unique combined chemical and electrochemical properties and are present together in the invention to support autonomic metabolic mechanisms that allow living tissues in a body to adjust serum anion gap levels and maintain their specific homeostatic acid-base equilibrium, lessening a body's dependence on minerals sacrificed from physiologically essential repositories in order to compensate for pH swings.

The formulation of this invention can be consumed orally in dosage units formed as tablets, capsules, gelcaps, caplets and other ingestible delivery mechanisms. These ingestible delivery mechanisms are enteric-coated to permit their transport through the stomach intact, becoming dissolvable in the small intestines. The formulation's inorganic compounds are control-released from their ingestible delivery mechanisms into the small intestines and are absorbed by the arterial blood supply as bioavailable active ingredients that assimilate into intracellular fluids, extracellular fluids and all other differentiated living tissues in a body.

The formulation also can be dissolved in a solution of United States Pharmacopeia (“USP”) purified pharmaceutical grade water and administered intravenously in standard dosage units. The intravenous routes of administration include both infusion and injection. Infusion (“drips”) introduces the formulation (dissolved in a solution of USP purified pharmaceutical grade water) into a vein. Injection (“sharps”) introduces the formulation (dissolved in USP purified pharmaceutical grade water) into a vein, an artery or other targeted differentiated living tissues in the human body.

### OBJECTS OF THE INVENTION

The first object of the present invention is to provide a base medicinal formulation that delivers the essential and indispensable electro-metallic base minerals magnesium and zinc to the arterial blood supply as sacrificial bio-anodes and bio-cathodes that influence alterations in the anion gap in intracellular fluids, extracellular fluids and all other differentiated living tissues in the human body. Magnesium and zinc provide unique efficacies to adjust serum anion gap levels and influence homeostatic acid-base equilibrium in the presence of low buffer and high buffer concentrations.

A second object of this invention is to provide a base medicinal formulation that delivers bicarbonate, a transitional base chemical of the metabolic process that buffers acid in support of pH balance in arterial blood plasma in the narrow alkaline range of 7.37 to 7.43. The invention's adjunctive bioavailable bicarbonate mitigates disruptions to homeostatic acid-base balance when bicarbonate production is insufficient, endogenous acid production (EAP) is excessive and the autonomic metabolic mechanisms of a body are overwhelmed.

A third object of the invention is to support autonomic metabolic mechanisms that allow intracellular fluids, extracellular fluids and all other differentiated living tissues in a body to maintain their specific homeostatic acid-base equilibrium, becoming either more base or less base, based on physiological demand and the introduction of the base medicinal formulation of this invention into the human body.

A fourth object of the invention is to provide this formulation in orally administered dosage units formed as tablets, capsules, gelcaps, caplets or other ingestible delivery mechanisms with an enteric coating that allows the formulation to be transported through the stomach intact, becoming dissolvable in the small intestines.

A fifth object of the invention is to provide for controlled-release of the formulation's inorganic compounds from their ingestible delivery mechanisms into the small intestines and absorption by the arterial blood supply as bioavailable active ingredients that assimilate into intracellular fluids, extracellular fluids and all other differentiated living tissues in a body.

A sixth object of the invention is to provide the formulation in a solution of United States Pharmacopeia (“USP”) purified pharmaceutical grade water, such that it may be administered intravenously in standard dosage units by infusion. Infusion (“drips”) introduces the formulation (dissolved in USP purified pharmaceutical grade water) into a vein.

A seventh object of the invention is to provide the formulation in a solution of USP purified pharmaceutical grade water, such that it may be administered intravenously in standard dosage units by injection. Injection (“sharps”) introduces the formulation (dissolved in USP purified pharmaceutical grade water) into a vein, an artery or other targeted differentiated living tissues in a body.

An eighth object of the invention is to provide the formulation dissolved in a solution of USP purified pharmaceutical grade water, such that it may be adopted for its clinical efficacy as a packaging medium for surgically implantable materials (i.e., sutures).

A ninth object of the invention is to reduce a body's dependency on resorbed alkaline minerals sacrificed from other physiologically essential repositories to compensate for pH swings when there is bicarbonate deficiency or endogenous acid production overwhelms the pathophysiological rate or other factors.

### DESCRIPTION OF THE PRIOR ART

In prior art, potassium bicarbonate and sodium bicarbonate were established as a means of delivering bioavailable water-soluble and oil-soluble vitamins and amino acid chelated minerals in an effervescent vitamin-mineral granule preparation which, when dissolved in water, produced a flavored, lightly carbonated drink. Ashmead, et al., U.S. Pat. No. 4,725,427 (Feb. 16, 1988). In the vitamin and mineral blend of their novel supplementation, the inventors included minerals selected from a group consisting of calcium, magnesium, iron, zinc, copper and manganese. That same year, a multi-mineral dietary supplement composition for oral administration was introduced as a means of delivering iron supplementation accompanied by bioavailable calcium and magnesium in order to prevent or treat iron deficiency. Briggs, et al., U.S. Pat. No. 4,752,479 (Jun. 21, 1988).

Potassium bicarbonate and sodium bicarbonate were revisited a decade later in tablet form as the means of delivering bicarbonate to the human body in order to neutralize the acidic wastes produced by the body as a result of metabolic activities. Whang, U.S. Pat. No. 5,914,130 (Jun. 22, 1999). The tablets provided an alternative to an earlier conceived concentrated alkaline booster solution additive which, itself, was purported to overcome perceived deficiencies posed by the use of water ionizer machines. Whang, U.S. Pat. No. 5,306,511 (1994). That alkaline booster solution additive consisted of potassium hydroxide and sodium hydroxide wherein the said invention disclosed a producible consumable alkaline drinking water when said additive was dissolved in a glass of distilled water. Id., Whang.

It is known that alkaline drinking water will raise pH in the cavity of the stomach. The stomach, maintaining a low pH (acidity) in order to digest food, reacts when alkaline water is ingested by producing more hydrochloric acid and correspondingly, additional bio-available bicarbonate by natural autonomic metabolic mechanisms. The bio-available bicarbonate travels to the intestines and is absorbed into the arterial blood supply. Enteric-coated tablets and other similar delivery mechanisms containing potassium bicarbonate and sodium bicarbonate bypass the digestive mechanisms of the stomach altogether, releasing their contents—the bicarbonate and alkaline derived minerals—directly into the small intestines, where they are absorbed into the arterial blood supply. Regardless of which form of oral delivery (liquid or solid) is preferred, the arterial blood supply is where bicarbonate supports healthy human metabolic physiology: by mitigating endogenous acid, by compensating for disturbances in arterial blood pH, and by reducing a body's dependency on resorbed alkaline minerals sacrificed from other physiologically essential repositories to compensate for those pH swings.

Healthy human metabolic physiology also requires adjusted serum anion gap levels inasmuch as an elevated anion gap indicates the presence of some degree of clinical metabolic dysfunction, more often than not a respiratory or metabolic acidosis. Magnesium (an alkaline earth metal) and zinc (a transition metal) possess indispensable anodic and cathodic properties that influence alterations in the anion gap and contribute to maintenance of specific homeostatic acid-base equilibrium in the differentiated living tissues of a human body. Green, U.S. pat. No. 9,986,752 (Jun. 5, 2018) (teaching the propensities of zinc to influence alterations in the anion gap).

Since water ionizers do not furnish either magnesium or zinc in their consumable water output, Whang did not include either of these minerals in his booster solution additive or his tablets for their supplementary value as minerals, much less for their electro-metallic properties. More specifically, Whang did not teach of the bio-electrochemical properties of magnesium and zinc in any of his compositions. Importantly, the Whang disclosures make no reference to the anion gap in the context of “pertaining to health” or in any other context. Whang raises no awareness (or proffers any clinical support) for the necessity to supply magnesium and zinc for unique bio-electrochemical (anodic and cathodic) properties that influence alterations in the anion gap based on the physiological demands of a patient or consumer.

Green, supra, introduced the concept that zinc functions as a bio-electrochemical sacrificial anode and cathode in the human body. Because zinc can donate electrons that will attract positive H⁺ ions (the anodic sacrifice) and protons that will attract negative H⁻ ions (the cathodic sacrifice), zinc is demonstrably clinically relevant to maintaining acid-base equilibrium in the differentiated living tissues of the human body. Zinc influences alterations in the anion gap in differentiated human environments by responding therapeutically to such instantaneous physiological demands of the body. See, for example, Six Patient Scenarios for a Co-Administration of Zinc (zinc carbonate) (Declaration under 37 CFR § 1.132 of Lawrence M. Green) (Clinical Scenarios “A” through “F”, demonstrating therapeutic strategies for correcting metabolic dysregulation in patients presenting with various clinical symptoms in the spectrum).

In the present invention, these teachings are expanded to include the parallel contributions of magnesium, which shares those specific electro-metallic qualities with zinc. Zinc (atomic number 30) and magnesium (atomic number 12) belong to a collection of conductive metals (that also include aluminum, iron, tin and lead) which are known to react moderately with acids. Beneficial in trace amounts, zinc and magnesium succeed in part because they maintain higher thresholds for toxicity in humans than these other metals. In some respects, zinc is chemically similar to magnesium: both elements exhibit only one normal oxidation state (+2), and the Zn²⁺ and Mg²⁺ ions are of similar size.

Zinc and magnesium are both compatible and complementary to each other in the formulation of this invention. They are compatible in that they both influence alterations in the anion gap in differentiated living tissues in low buffer concentration environments by donating electrons that will attract positive H⁺ ions from said living tissues, sacrificing themselves in the process of making said living tissues more base (alkaline) based on physiological demand. In high buffer concentration environments, both magnesium and zinc influence alterations in the anion gap in differentiated living tissues by donating protons that will attract negative H⁻ ions from said living tissues, sacrificing themselves in the process of making said living tissues less base based on physiological demand. Zinc and magnesium also behave complementary to each other in influencing acid-base equilibrium: zinc reacts more efficiently in high sodium environments whereas magnesium is more efficient in low sodium (or sodium-free) environments.

As has been explained in this disclosure, the formulation of the present invention, consisting of potassium bicarbonate, sodium bicarbonate, magnesium carbonate and zinc carbonate, is designed to influence alterations in the anion gap and to support the specific homeostatic acid-base equilibrium in the differentiated living tissues of a human body. The clinical efficacies obtainable from the organic compounds presented together in this invention are comparable to, and a natural evolution from, Green, supra, a liquid formulation consisting of potassium hydroxide, sodium hydroxide, magnesium carbonate hydroxide, zinc carbonate and United States Pharmacopeia water. The specific combined organic compounds of this invention are introduced, and advocated for in this disclosure, to provide practitioners with new clinically appropriate treatment modalities in trauma and acute care settings—alternatives to oral ingestion —in order to accommodate the exigencies of their patients (i.e., vomiting, inability to swallow, loss of consciousness).

To this inventor's knowledge, and further, based on information and belief, no formulations in the art prior to Green, supra, were capable of achieving the physiological efficacies and outcomes advocated by Green to adjust serum anion gap levels and mitigate metabolic dysregulation and its foreseeable health consequences. Quite simply, whereas earlier formulations in the art do not benefit from magnesium and zinc's electro-metallic properties as sacrificial bio-anodes and bio-cathodes, it follows that neither do those who consume them. These particular attributes of the present invention overcome a major deficiency in the prior art of delivering alkaline minerals for the purpose of supporting acid-base equilibrium by virtue of this invented formulation's delivery of superior results, tangibly evident in the context of supporting clinical aspects of human health wholly unanticipated by said prior art.

### DESCRIPTION OF PREFERRED EMBODIMENT AND BEST MODE OF THE INVENTION

The inorganic compounds embodying this invention in combination—potassium bicarbonate [KHCO₃⁻], sodium bicarbonate [NaHCO₃⁻], magnesium carbonate [MgCO₃] and zinc carbonate [ZnCO₃]—are formulated within specific ranges expressed in milligrams and percentages. For dosage units formed as tablets, capsules, gelcaps, caplets and other ingestible delivery mechanisms, the specific ranges are: between 3.0 and 90.0 milligrams of potassium bicarbonate [KHCO₃⁻]; between 1.0 and 10.0 milligrams of sodium bicarbonate [NaHCO₃⁻]; trace/not greater than, of magnesium carbonate [MgCO₃]; and trace/not greater than, of zinc carbonate [ZnCO₃]. For standard dosage units of intravenous administration (after dissolution in a solution of United States Pharmacopeia (“USP”) purified pharmaceutical grade water), the specific ranges are: between 1.0% and 3.0% of potassium bicarbonate [KHCO₃]; between 0.03% and 0.9% of sodium bicarbonate [NaHCO₃⁻]; trace/not greater than, of magnesium carbonate [MgCO₃]; and, trace/not greater than, of zinc carbonate [ZnCO₃]

This invention, described in sufficient detail in other sections of this disclosure, is formulated purposefully for ease of administration and contemplates being delivered to the arterial blood supply, the venal blood supply and other targeted differentiated living tissues in the human body employing a variety of methodologies that will accommodate clinical exigencies posed by both patients and consumers. The methodologies include, but are not limited to, delivery by enteric-coated tablets, capsules, gelcaps, caplets and other ingestible oral delivery mechanisms enclosing controlled-release agents, intravenous administration by infusion (drips) and intravenous administration by injection (sharps). In the case of intravenous administration by infusion or injection, the formulation of this invention is dissolved in a solution of United States Pharmacopeia (USP) purified pharmaceutical grade water. In addition, the formulation in solution may be adopted for its clinical efficacy as a packaging medium for surgically implantable materials (i.e., sutures).

Given the parameters described above, the formulation can be adjusted in order to increase or decrease the amount of bicarbonate relative to amounts of either potassium or sodium based on the clinical needs of patients and the pathophysiological susceptibilities of consumers. For example, patients suffering from chronic kidney disease resulting from diabetes or other metabolic dysfunctions may be more susceptible to hyperkalemia (elevated serum potassium). A patient with end-stage renal failure who is on dialysis must avoid potassium altogether. Patients who have congestive heart failure and other degenerative kidney and liver diseases can present in acute care settings with hyponatremia (low serum sodium). Conversely, consumers who have hypertension or other disease states within the metabolic syndrome generally are advised to limit their sodium consumption. In all embodiments of this invention, however, the trace amounts of magnesium carbonate and zinc carbonate as described herein are maintained for their positive influence to adjust serum anion gap levels in patients and consumers.

The claims of this invention are:

1. A base medicinal formulation consisting of the inorganic compounds: potassium bicarbonate [KHCO₃⁻], sodium bicarbonate [NaHCO₃⁻], magnesium carbonate [MgCO₃] and zinc carbonate [ZnCO₃].

2. The base medicinal formulation of claim 1, wherein the unique combined chemical and electrochemical properties of its inorganic compounds—potassium bicarbonate [KHCO₃⁻], sodium bicarbonate [NaHCO₃⁻], magnesium carbonate [MgCO₃] and zinc carbonate [ZnCO₃]—influence alterations in the anion gap and maintain homeostatic acid-base balance in arterial blood plasma in the narrow healthy alkaline range of 7.37 to 7.43 pH in support of autonomic metabolic mechanisms that allow intracellular fluids, extracellular fluids and all other differentiated living tissues in the human body to maintain their specific homeostatic acid-base equilibrium.

3. The base medicinal formulation of claim 2, wherein the unique combined chemical and electrochemical properties of its inorganic compounds—potassium bicarbonate [KHCO₃⁻], sodium bicarbonate [NaHCO₃⁻], magnesium carbonate [MgCO₃] and zinc carbonate [ZnCO₃]—influence alterations in the anion gap and maintain homeostatic acid-base balance in arterial blood plasma in the narrow healthy alkaline range of 7.37 to 7.43 pH when bicarbonate production is insufficient, or endogenous acid production (EAP) is excessive and a body is sacrificing resorbed alkaline minerals from other physiologically essential repositories in order to compensate for pH swings (imbalances) caused by EAP or other factors.

4. The base medicinal formulation of claim 3, wherein potassium [K] in the potassium bicarbonate [KHCO₃⁻] is dispersed through the arterial blood supply and utilized by a body based on physiological demand, and the bicarbonate [HCO₃⁻], a transitional base chemical of the metabolic process, buffers excess acid in the arterial blood supply by dissociating into carbonic acid (H₂CO₃), which further dissociates into water (H₂0) and carbon dioxide (CO₂), which is exhaled.

5. The base medicinal formulation of claim 3, wherein sodium [Na] in the sodium bicarbonate [NaHCO₃⁻] is dispersed through the arterial blood supply and utilized by a body based on physiological demand, and the bicarbonate [HCO₃⁻], a transitional base chemical of the metabolic process, buffers excess acid in the arterial blood supply by dissociating into carbonic acid (H₂CO₃), which further dissociates into water (H₂O) and carbon dioxide (CO₂), which is exhaled.

6. The base medicinal formulation of claim 3, wherein the carbonate [CO₃⁻₂] in the magnesium carbonate [MgCO₃] either remains a salt in the arterial blood supply based on physiological demand, or is dissolved in carbonic acid, which dissociates into water (H₂O) and carbon dioxide (CO₂), which is exhaled, and the magnesium [Mg] is dispersed through the arterial blood supply to low sodium environments in a body where it acts as a sacrificial bio-anode and bio-cathode that influences alterations in the anion gap, either by donating electrons that will attract positive H+ions from differentiated living tissues (the anodic sacrifice) or by donating protons that will attract negative H⁻ ions from differentiated living tissues (the cathodic sacrifice) that allow said living tissues to become more or less base based on physiological demand.

7. The base medicinal formulation of claim 3, wherein the carbonate [ CO₃-₂] in the zinc carbonate [ZnCO₃] either remains a salt in the arterial blood supply based on physiological demand, or is dissolved in carbonic acid, which dissociates into water (H₂O) and carbon dioxide (CO₂), which is exhaled, and the zinc [Zn] is dispersed through the arterial blood supply to high sodium environments in a body where it acts as a sacrificial bio-anode and bio-cathode that influences alterations in the anion gap, either by donating electrons that will attract positive H⁺ ions from differentiated living tissues (the anodic sacrifice) or by donating protons that will attract negative H⁻ ions from differentiated living tissues (the cathodic sacrifice) that allow said living tissues to become more or less base based on physiological demand.

8. The base medicinal formulation of claim 3, wherein the inorganic compounds, potassium bicarbonate [KHCO₃⁻], sodium bicarbonate [NaHCO₃⁻], magnesium carbonate [MgCO₃] and zinc carbonate [ZnCO₃], are present in the formulation within the following ranges per dosage unit: between 3.0 and 90.0 milligrams of potassium bicarbonate [KHCO₃⁻]; between 1.0 and 10.0 milligrams of sodium bicarbonate [NaHCO₃⁻]; trace/not greater than, of magnesium carbonate [MgCO₃]; and, trace/not greater than, of zinc carbonate [ZnCO₃].

9. The base medicinal formulation of claim 8, wherein the methods of oral administration of said formulation include dosage units formed as tablets, capsules, gelcaps, caplets and other ingestible delivery mechanisms.

10. The base medicinal formulation of claim 9, wherein said ingestible delivery mechanisms are enteric-coated, permitting said delivery mechanisms to be transported through the stomach intact and become dissolvable in the small intestines.

11. The base medicinal formulation of claim 10, wherein said ingestible delivery mechanisms include a controlled-release agent for absorption of the formulation's inorganic compounds from the small intestines into the arterial blood supply as bioavailable active ingredients that assimilate into intracellular fluids, extracellular fluids and all other differentiated living tissues in a body.

12. The base medicinal formulation of claim 11 which, when swallowed in said forms of ingestible delivery mechanisms with a glass of water, is transported through the stomach intact and becomes dissolvable in the small intestines wherein the formulation's inorganic compounds, potassium bicarbonate [KHCO₃⁻], sodium bicarbonate [NaHCO₃⁻], magnesium carbonate [MgCO₃] and zinc carbonate [ZnCO₃], are control-released from said delivery mechanisms and are absorbed by the arterial blood supply as bioavailable active ingredients.

13. The base medicinal formulation of claim 3, wherein the inorganic compounds, potassium bicarbonate [KHCO₃⁻], sodium bicarbonate [NaHCO₃⁻], magnesium carbonate [MgCO₃] and zinc carbonate [ZnCO₃], present in the formulation within the ranges of between 1.0% and 3.0% of potassium bicarbonate [KHCO₃]; between 0.03% and 0.9% of sodium bicarbonate [NaHCO₃⁻]; trace/not greater than, of magnesium carbonate [MgCO₃]; and, trace/not greater than, of zinc carbonate [ZnCO₃] per dosage unit, are dissolved in a solution of United States Pharmacopeia (USP) purified pharmaceutical grade water for intravenous administration by infusion (commonly referred to as “drips”), which introduces said formulation into a vein.

14. The base medicinal formulation of claim 3, wherein the inorganic compounds, potassium bicarbonate [KHCO₃⁻], sodium bicarbonate [NaHCO₃⁻], magnesium carbonate [MgCO₃] and zinc carbonate [ZnCO₃], present in the formulation within the ranges of between 1.0% and 3.0% of potassium bicarbonate [KHCO₃]; between 0.03% and 0.9% of sodium bicarbonate of sodium bicarbonate [NaHCO₃⁻]; trace/not greater than, of magnesium carbonate [MgCO₃]; and, trace/not greater than, of zinc carbonate [ZnCO₃] per dosage unit, are dissolved in a solution of United States Pharmacopeia (USP) purified pharmaceutical grade water for intravenous administration by injection (commonly referred to as “sharps”), which introduces said formulation into a vein, an artery or other targeted differentiated living tissues in the human body.

15. The base medicinal formulation of claim 8, wherein the formulation, dissolved in a solution of United States Pharmacopeia (USP) purified pharmaceutical grade water may be adopted for its clinical efficacy as a packaging medium for surgically implantable materials (i.e., sutures).
